(19) United States
(12) Patent Application Publication
(10) Pub. No.: US 2007/0173479 A1
(43) Pub. Date: Jul. 26, 2007

(54) Methods Using Sulodexide for the Treatment of Bladder Disease

(75) Inventor: Enrique Poradosu, Brookline, MA (US)

Correspondence Address: JONES DAY, 222 EAST 41ST ST, NEW YORK, NY, 10017, US

(73) Assignee: KERYX BIOPHARMACEUTICALS, INC., New York, NY (US)

(21) Appl. No.: 10/582,587

(22) PCT Filed: Dec. 9, 2004
(86) PCT No.: PCT/US04/41394

§ 371 Date: Jun. 9, 2006

Related U.S. Application Data
(60) Provisional application No. 60/528,470, filed on Dec. 10, 2003.

(51) Int. Cl. A61K 31/737; A61K 31/727

(52) U.S. Cl. 514/54; 514/56

(57) ABSTRACT

The present invention concerns methods for the treatment of bladder related diseases and, in particular, inflammatory bladder diseases such as interstitial cystitis, by administration of sulodexide.

This application claims benefit under 35 U.S.C. § 119(e) of U.S. Provisional Application Ser. No. 60/528,470 filed Dec. 10, 2003, which is incorporated herein by reference in its entirety.

### FIELD OF THE INVENTION

The present invention concerns methods for the treatment of bladder related diseases and, in particular, inflammatory bladder diseases such as interstitial cystitis, by administration of sulodexide.

### BACKGROUND OF THE INVENTION

Interstitial cystitis (“IC”) is a chronic sterile inflammatory disease of the bladder characterized by urinary frequency, urgency, nocturia and pelvic pain. Although there are many theories concerning its causes, the etiology of the disease is poorly understood. For a general discussion of the pathology of IC, see, e.g., Rosamilia et al., 2003, Int. J. Urol. 10 (Supp. 1):S11-S15.

Several pathophysiological mechanisms have been proposed in the past few years in an attempt to explain its causes, including epithelial dysfunction, activation of mast cells, neurogenic inflammation, autoimmunity and occult infection. Evidence indicates that IC is a heterogeneous syndrome of two subtypes, classic and non-ulcer disease, which represent different mechanisms. The diagnosis of interstitial cystitis is very problematic as many gynecological and urological conditions have a similar manifestation as interstitial cystitis. Diagnosis is currently made by clinical and cytoscopic evaluation with hydrodistension, often with biopsy once other disorders are excluded. An anti-proliferative factor in the urine has shown initial promising results as a marker for IC; however, further validation is required (Keay et al., 2001, Urology 57 (6 Supp. 1):9-14).

Since the symptoms of IC vary so much in their manifestation and severity, some researchers believe that IC represents several separate diseases. In the general population, diagnosis of IC is based on the presence of urgency and frequency of urination, as well as the presence of pelvic/bladder pain. Cytoscopic evidence of bladder wall inflammation, including ulcers or glumerilations, are present in about 90% of patients with IC. Currently, there is no single for IC nor any consistent manner to predict which patient will respond best to which treatment. Symptoms may disappear spontaneously without explanation or sometimes coincide with change of diet or treatment. However, symptoms may reappear after days, weeks, months or years without any known physiological explanation.

As the cause(s) of IC are unknown, current treatments are aimed at relieving symptoms associated with IC. A common treatment concerns various manipulations of the bladder, such as bladder distension which may be helpful due to the increased capacity of the bladder and due to the interference with pain signals transmitted by the nerves in the bladder. Another manipulation is bladder instillation (“bladder wash/or bath”) where the bladder is filled with a solution, which solution is held for varying periods of time (averaging 10 to 15 minutes), and then the bladder is emptied.

Currently the only drug approved by the Federal Drug Administration of the United States (“FDA”) for bladder instillation is RIMSO-50™, which is dimethylsulfoxide (DMSO). DMSO may work by effectively reducing inflammation and blocking pain, as well as by preventing muscle contractions that cause pain, frequency and urgency. However, a bladder instillation, in general, and by DMSO, has several drawbacks. For example, the patient has to go into a physician's office for the treatment every 6 to 8 weeks, the patient must be catheterized, and DMSO has a significant garlic-like taste and odor that exudes from the skin that may last up to 72 hours after treatment.

In 1996, pentosan polysulfate sodium (Elmiron™) (Ortho-McNeil, Raritan, N.J.) was the first drug approved for oral treatment of IC by the FDA. However, the drug's effects are only apparent two to four months after the start of treatment with regard to relief from pain, and only apparent six months after the start of treatment with respect to a decrease in urinary frequency. See, also, Nickel et al., 2001, Urology 57 (6 Suppl. 1): 122-123; Hanno, 1997, Urology 49 (5A Suppl.):93-99 for discussions on the use of Elmiron™ for the treatment of IC.

Heparin has also been used to treat patients with IC (Lose et al., 1986, Scand. J. Urol. Nephrol. 19:27-29; Parsons et al., 1994, Br. J. Urol. 73:504-507).

Other oral medications mentioned in connection with IC are aspirin and ibuprofen, which may be the first line of defense against discomfort as commonly used pain relievers. For recent developments in the management of interstitial cystitis see, e.g., Bouchelouche et al., 2003, Curr. Opin. Urol. 13:309-313.

In addition to the above medical treatments, some IC symptoms are alleviated by a decrease in the consumption of foods having a high acid content, such as alcohol, tomatoes, spices, chocolate, caffeine and citrus juice. As is evident, there is a current need for an effective treatment of IC and its symptoms and complications.

Glycosaminoglycans, such as heparin, are routinely used in anticoagulant and antithrombotic therapies. Sulodexide is a glycosaminoglycan (GAG) of natural original extracted from mammalian intestinal mucosa and possessing an anticoagulant activity and a sulfation degree lower than that of heparin, as shown by Radhakrishnamurthy et al., 1978, Atherosclerosis 31:217-229. The preparation of Sulodexide is described in U.S. Pat. No. 3,936,351.

Sulodexide is marketed in Europe under the trademark VESSEL DUE F® and is prescribed for the treatment of vascular pathologies with thrombotic risk such as peripheral occlusive arterial disease (POAD), healing of venous leg ulcers, and intermittent claudication (Harenberg, 1998, Med. Res. Rev. 18:1-20, Crepaldi et al., 1990, Atherosclerosis 81:233), cardiovasculopathies (Tramarin et al., 1987, Medical Praxis 8:1), cerebrovasculopathies (Sozzi, 1984, Eur. Rev. Med. Pharmacol. Sci. 6:295), and venous pathologies of the lower limbs (Cospite et al., 1992, Acta Therapeutica, 18:149, (1992).

Kanway et al., 1988, Sem. Nephrol. 5:307 and Groggel et al., 1988, Kidney Int. 33:517, provide evidence of the probable role of glycosaminoglycans in helping the integrity and the functioning of the renal cells.

Canfield, et al., 1978, Lab. Invest. 39:505 and Jensen, 1997, Diabetes 46 (Suppl. 2):S98-S100 have shown a decrease of membranal glycosaminoglycans in conditions of diabetic nephropathy. This decrease may be mediated by decreased heparan sulfate production and/or sulfation (Raats et al., 2000, Kidney Int. 57:385-400).

U.S. Pat. No. 5,236,910 discloses the use of glycosaminoglycans for the treatment of diabetic nephropathy and diabetic neuropathy. U.S. Pat. No. 5,496,807 discloses a method of treatment of diabetic nephropathy by the administration of sulodexide.

Citation or identification of any reference in this section or in any other section of this application shall not be construed as an admission that such reference is available as prior art to the present invention.

### SUMMARY OF THE INVENTION

The present invention is directed to a method of preventing, reducing or eliminating a symptom or complication of bladder inflammation such as interstitial cystitis (IC) comprising, administering sulodexide to a subject in need of such treatment in an amount effective to inhibit, reduce or eliminate one or more symptoms or complications of interstitial cystitis.

The present invention is further directed to the use of sulodexide in the preparation of a medicament for the prevention, reduction or elimination of a symptom or complication of interstitial cystitis.

The present invention is also directed to a pharmaceutical composition for the prevention, reduction or elimination of a symptom or complication of interstitial cystitis, which composition comprises, as an active ingredient, sulodexide, together with a pharmaceutically acceptable carrier.

In an embodiment of the invention, the subject is a mammal, preferably a human. In a preferred embodiment, the subject is not diabetic. In yet another preferred embodiment, the subject is not infected with a human immunodeficiency virus (HIV). In yet another embodiment, the subject does not have kidney disease. In a more preferred embodiment of the invention, the subject is a human subject that is not diabetic, does not have kidney disease, and is not infected with HIV.

In one embodiment, sulodexide is administered in an amount of 10-1000 mg/day, preferably 50-500 mg/day, more preferably 100-400 mg/day.

### DETAILED DESCRIPTION OF THE INVENTION

The present invention encompasses a method for the prevention, reduction or elimination of a symptom or complication of IC by administration to a patient, in need of thereof, an effective amount of sulodexide.

The term “sulodexide” in the context of the invention refers to a composition comprising from about 60% to about 90% iduronylglycosaminoglycan sulfate and between about 10% to about 40% dermatan sulfate. This term in the context of the present invention refers also to a pharmaceutically acceptable salt, solvate, hydrate, and clathrate of sulodexide.

Preferably, sulodexide comprises about 80% iduronylglycosaminoglycan sulfate (IGGS), which is a fast-moving heparin fraction, and about 20% dermatan sulfate. The fast moving component, which is determined by its electrophoretic mobility in the barium-propanediamine system, is found in commercial heparin along with a slower moving component. IGGS has a low to medium molecular weight of about 7 kD and a lower anticoagulant activity than the slow moving heparin fraction and unfractionated heparin. Compared to heparin, IGGS has the same dimeric component but with lower amounts of iduronic acid-2-O-sulfate and a different amount of glucosamine N-acetylated-glucuronic acid dimer.

The term “prevention, reduction or elimination of a symptom or complication of interstitial cystitis” in the context of the present invention refers to at least one of the following: prevention of IC before it occurs, for example, in patients that suffered in the past from acute IC but are now in a period of remission; elimination of established IC (as determined by, for example, the return of normal urinary function); elimination of pelvic pain associated with IC; reduction of an undesired symptom of the disease as manifested by a decrease in the severity of an existing condition of IC; elimination or reduction of one or more medications used in treating the subject. The reduction in the undesired symptom may be determined by, for example, improvement in urinary function (reduced urgency and frequency of urination) as compared to before treatment, even if some of the undesired symptom remains at a lower, more acceptable level. Such remediation may be evident as a delay in the onset of severe urinary distortion or in a decrease in the rate of deterioration of urinary distortion, or decrease in the level of pelvic pain.

Since interstitial cystitis is often accompanied by other non bladder-related symptoms, such as pelvic discomfort, backache, dizziness, chest pain, aches and joint pain, abdominal cramping, nausea, heart pounding and headaches, reduction in at least one of these symptoms is encompassed by the term “prevention reduction or elimination of a symptom or complication of interstitial cystitis”.

Vulvadynia is a syndrome marked by pain in the vagina or vulva and is the fourth most common IC-associated condition. The prevention or reduction or elimination of a symptom or complication of IC may include also treatment, i.e., prevention, alleviation or reduction of vulvadynia, whether associated with other complications of interstitial cystitis or not, or whether vulvadynia is the sole or main manifestation of IC. See, Erickson et al., 2001, J. Urol. 166:557-561 for a discussion of non-bladder related symptoms in patients with interstitial cystitis.

The method of administration, according to the present invention, may be oral, mucosal, parenteral, intramuscular or transdermal. The dosage of the active ingredient will vary considerably depending on the mode of administration, the patient's age, weight and the patient's general condition, as well as the severity of the disease, and can be determined by standard clinical techniques. In addition, animal assays may optionally be employed to help identify optimal dosage ranges. The precise dose to be employed should be decided according to the judgment of the practitioner and each patient's circumstances.

Preferably the pharmaceutical composition is in a form acceptable for oral administration. Because of their ease of administration, tablets and capsules are preferred and represent the most advantageous oral dosage unit form where solid pharmaceutical excipients are employed. If desired, tablets may be coated by standard aqueous or non-aqueous techniques.

Preferably, the oral pharmaceutical composition used in the methods of the invention may be administered in a single or divided dosage from to 1 to 4 times per day.

The pharmaceutical composition can comprise any commercially available form of sulodexide, for example, VESSEL DUE F® commercially available form Alpha Wassermann, SpA in Italy. Preferred solid dosage forms of the pharmaceutical compositions are tablets or capsules which are coated or uncoated, and the preferred dosage forms range from about 10 mg per day to about 1,000 mg per day, preferably from about 50 mg to about 500 mg per day, more preferably from about 100 mg to about 400 mg per day. Preferably 400 mg per day is administered.

Where administration is parenteral (intramuscular or transdermal) and the active ingredient is sulodexide, the dosage should be in the range of 50-400 mg/day, preferably 50-100 mg/day.

Dosage Forms

Pharmaceutical compositions used in the method of the present invention suitable for oral administration may be presented as discrete pharmaceutical unit dosage forms, such as capsules, cachets, soft elastic gelatin capsules, tablets, caplets, or aerosol sprays, each containing a predetermined amount of the active ingredient, such as a powder or granules, or as a solution or a suspension in an aqueous liquid, a non-aqueous liquid, an oil-in-water emulsion, or a water-in-oil liquid emulsion. Dosage forms such as oil-in-water emulsions typically comprise surfactants such as anionic phosphate ester or lauryl sulfates, but other types of surfactants such as cationic or nonionic surfactants may be used in the compositions of the present invention. See generally, Remington's Pharmaceutical Sciences, 18^{th} ed., Mack Publishing, Easton Pa. (1990).

Pharmaceutical compositions of the present invention suitable for oral administration may be formulated as a pharmaceutical composition in a soft elastic gelatin capsule unit dosage form by using conventional methods well known in the art. See, e.g., Ebert, 1977, Pharm. Tech., 1(5): 44-50. Pharmaceutical compositions in the form of capsules or tablets coated by an enterosoluble gastro resistant film and which contains a lyophilisate consisting of glycosaminoglycan, a thickening agent, and a surfactant have been previously described in U.S. Pat. No. 5,252,339, which is incorporated herein by reference in its entirety.

Soft elastic gelatin capsules have a soft, globular gelatin shell somewhat thicker than that of hard gelatin capsules, wherein a gelatin is plasticized by the addition of plasticizing agent, e.g., glycerin, sorbitol, or a similar polyol. Varying the type of gelatin used and the amounts of plasticizer and water may change the hardness of the capsule shell. The soft gelatin shells may contain a preservative, such as methyl and propylparabens and sorbic acid, to prevent the growth of fungi. The active ingredient may be dissolved or suspended in a liquid vehicle or carrier, such as vegetable or mineral oils, glycols, such as polyethylene glycol and propylene glycol, triglycercides, surfactants, such as polysorbates, or a combination thereof.

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, microcrystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g. Nos. 2208, 2906, 2910), microcrystalline cellulose, and mixtures thereof.

Suitable forms of microcrystalline cellulose include, but are not limited to, the materials sold as AVICEL® PH-101, AVICEL® PH-103 AVICEL® RC-581, AVICEL® PH-105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL® RC-581. Suitable anhydrous or low moisture excipients or additives include AVICEL® PH-103 and Starch 1500 LM.

Examples of fillers suitable for use in the pharmaceutical compositions and dosage forms disclosed herein include, but are not limited to talc, calcium carbonate (e.g. granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Pharmaceutical stabilizers may also be used to stabilize the compositions of the invention. Acceptable stabilizers include but are not limited to L-cysteine hydrochloride, glycine hydrochloride, malic acid, sodium metabisulfite, citric acid, tartaric acid and L-cysteine dihydrochloride.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally after the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

In addition to the common dosage forms set out above, the compounds of the present invention may also be administered by controlled release means or delivery devices that are well known to those of ordinary skill in the art, such as those described in U.S. Pat. Nos.: 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566.

These pharmaceutical compositions can be used to provide slow or controlled-release of one or more of the active ingredients therein using, for example, hydropropylmethyl cellulose, other polymer matrices, gels, permeable membranes, osmotic systems, multilayer coatings, microparticles, liposomes, microspheres, or the like, or a combination thereof to provide the desired release profile in varying proportions. Suitable controlled-release formulations known to those of ordinary skill in the art, including those described herein, may be readily selected for use with the pharmaceutical compositions of the invention. Thus, single unit dosage forms suitable for oral administration, such as tablets, capsules, gelcaps, caplets, and the like, that are adapted for controlled-release are encompassed by the present invention.

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

Co-Administration

The method of treatment of the present invention may also include co-administration of another therapeutically effective agent for the treatment of a bladder disease, such as IC, together with the administration of sulodexide. Additionally, the method may also include co-administration of another therapeutic agent for the treatment of another disease(s) afflicting the subject. Examples of such agents that can be co-administered with the active ingredients are pain-relievers and other drugs used for the treatment of IC, such as pentosan polysulfate sodium (Elmiron™); amitriptyline chlorohydrate, which is a tricyclic antidepressant; anti-histamines, such as hydroxyzine and cromolyn sodium; anti-inflammatories, such as steroids; heparin; and DMSO.

The following series of examples are presented by way of illustration and not by way of limitation on the scope of the present invention.

### EXAMPLES

### Treatment of Interstitial Cystitis by Sulodexide

Clinical Protocol

1. First visit-screening/inclusive criteria for diagnosis

The first meeting is held at the urology clinic, and medical history and physical examination are carried out with emphasis on the following inclusion criteria:

a. Age>18

b. Presence of irritative urinary symptoms for more than 6 months

c. Negative urine culture

d. Negative urinary cytology

e. Typical cystoscopic findings under anesthesia during the last 3 years

f. Bladder biopsies suggestive for IC

g. Failed previous treatment with either DMSO, or elatrol or elmirone

h. Negative urine tests for pregnancy for women in childbearing age

i. Fill index questionnaire

Treatment Regime

Once diagnosed as suffering from IC, the patient is administered sulodexide VESSEL DUE F®) in an amount of 200 mg daily for 12 weeks, by twice a day administration of 100 mg.

2. Treatment follow-up

Patients are assessed again for medical history and asked to fill a voiding diary for three days, at week 23 and at week 29 from start of treatment.

A typical urodynamic study consists of:

1. Patient is usually asked to empty the bladder before the procedure takes place.

2. The bladder may be imaged with x-rays or ultrasound and the kidneys may also be imaged.

3. Two small catheters (tubes) are inserted through the normal passage to the bladder.

4. One of these is used to run liquid into the bladder to measure the bladder's capacity and the other tube will measure the bladder pressure.

5. The patient will be asked to tell the operator when he first gets sensation of needing to pass urine, feeling the bladder is full, and when he cannot hold anymore.

A urodynamic study is performed at the beginning of treatment (between first and second visit) and the end of treatment. The voiding diary is completed four times during treatment, on visit 2 (pretreatment), visit 3 (after 12 weeks), visit 4 (end of treatment) and visit 5 (post treatment).

The following questionnaire is filled out to assist in diagnosing the patient.

Interstitial Cystitis (IC) Symptom and Problem Questionnaire*

Identifying IC

To help your physician determine if you have IC, please put a check mark next to the most appropriate response to each of the questions shown below. Then, add up the numbers to the left of the check marks and write the total below.

[image]

[image]

[image]

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled. Such modifications are intended to fall within the scope of the appended claims.

All references, patent and non-patent, cited herein are incorporated herein by reference in their entireties and for all purposes to the same extent as if each individual publication or patent or patent application was specifically and individually indicated to be incorporated by reference in its entirety for all purposes.

What is claimed:

1. A method for preventing, reducing or eliminating a symptom or complication of interstitial cystitis comprising: administering to a subject, in need thereof, sulodexide in an amount effective to inhibit, reduce or eliminate one or more symptoms or complications of interstitial cystitis.

2. The method of claim 1 in which the subject is a human subject.
